(19) United States
(12) Patent Application Publication
(10) Pub. No.: US 2024/0049945 A1
(43) Pub. Date: Feb. 15, 2024

(54) SUCTION VALVE FOR AN ENDOSCOPE

(71) Applicant: Boston Scientific Scimed, Inc., Maple Grove, MN (US)

(72) Inventor: Colby Harris, Norfolk, MA (US)

(73) Assignee: Boston Scientific Scimed, Inc., Maple Grove, MN (US)

(21) Appl. No.: 18/446,204

(22) Filed: Aug. 8, 2023

Related U.S. Application Data
(60) Provisional application No. 63/396,437, filed on Aug. 9, 2022.

(51) Int. Cl. A61B 1/00; A61B 1/015

(52) U.S. Cl. CPC A61B 1/00068; A61B 1/015

(57) ABSTRACT

Devices, systems, and methods for a suction valve assembly for a medical device. The suction valve assembly may include a valve well and a valve stem. The valve stem may include a first seal, a second seal, and a third seal. The valve stem may include a first opening and a second opening fluidly coupled to one another via a lumen. The valve well may include a proximal opening, a distal opening, and an intermediate opening. The valve stem may be configured to translate within the valve well to adjust between a first configuration in which the third seal is a barrier between the intermediate opening and the distal opening and the lumen is fluidly isolated from the intermediate opening and a second configuration in which the first seal is a barrier between the proximal opening and the intermediate opening and the lumen is isolated from the proximal opening.

### CROSS REFERENCE TO RELATED APPLICATIONS

This application claims the benefit of U.S. Provisional Patent Application Ser. No. 63/396,437, filed Aug. 9, 2022, titled SUCTION VALVE FOR AN ENDOSCOPE, which is incorporated herein by reference.

### TECHNICAL FIELD

This disclosure relates generally to valve assemblies and methods, and particularly to suction valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, A suction valve assembly for a medical device may comprising a valve well having a proximal opening, a distal opening, and an intermediate opening positioned between the proximal opening and the distal opening, a valve stem configured to translate within the valve well, the valve stem comprising a first opening, a second opening, a lumen extending from the first opening to the second opening, a first seal extending circumferentially around the valve stem at a location proximal of the first opening, a second seal extending circumferentially around the valve stem at a location distal of the first opening, and a third seal extending circumferentially around the valve stem at a location distal of the second seal. The valve stem may have a first configuration in the valve well in which the second seal is a barrier between the proximal opening and the intermediate opening, the third seal is a barrier between the intermediate opening and the distal opening, and the lumen is fluidly isolated from the intermediate opening, and the valve stem may have a second configuration in the valve well in which the first seal is proximal the intermediate opening and the lumen is fluidly isolated from the proximal opening and the first seal is a barrier between the proximal opening and the intermediate opening.

Alternatively or additionally to any of the examples above, when the valve stem is in the first configuration, the proximal opening and the distal opening are in fluid communication through the lumen of the valve stem.

Alternatively or additionally to any of the examples above, when the valve stem is in the second configuration, the intermediate opening and the distal opening are in fluid communication through the lumen of the valve stem.

Alternatively or additionally to any of the examples above, when the valve stem is in the first configuration, the third seal is distal of the intermediate opening and the distal opening is fluidly isolated from the intermediate opening.

Alternatively or additionally to any of the examples above, the second seal and the third seal are configured to contact an inner wall of the valve well when the valve stem is in the first configuration.

Alternatively or additionally to any of the examples above, the first opening is a radial opening in the valve stem and the second opening is an axial opening in the valve stem.

Alternatively or additionally to any of the examples above, the valve well may further comprises a main body defining the intermediate opening and the distal opening, and a collar couplable to the main body, the collar defining the proximal opening.

Alternatively or additionally to any of the examples above, the first seal is configured to engage the collar when the valve stem is in the first configuration.

Alternatively or additionally to any of the examples above, the suction valve assembly may further include a cap coupled to the valve stem, a bias mechanism extending between the cap and the valve well, and wherein the bias mechanism is configured to bias the valve stem to the first configuration.

Alternatively or additionally to any of the examples above, the cap, the bias mechanism, the valve stem, and the valve well are configured such that actuation of the cap in a direction opposite a direction of a bias force of the bias mechanism is configured to adjust the valve stem from the first configuration to the second configuration.

Alternatively or additionally to any of the examples above, the valve stem may comprise: a first circumferentially recessed portion at which the first seal is located, a second circumferentially recessed portion at which the second seal is located, and a longitudinal recessed portion extending between the first circumferentially recessed portion and the second circumferentially recessed portion.

Alternatively or additionally to any of the examples above, the valve stem may comprise: a first circumferentially recessed portion at which the first seal is located, a second circumferentially recessed portion at which the second seal is located, a third circumferentially recessed portion at which the second seal is located, a first longitudinal recess extending between the first circumferentially recessed portion and the second circumferentially recessed portion, and a second longitudinal recess extending between the second circumferentially recessed portion and the third circumferentially recessed portion.

Alternatively or additionally to any of the examples above, a material forming one or more of the first seal, the second, seal, and the third seal extends within one or both of the first longitudinal recess and the second longitudinal recess.

Alternatively or additionally to any of the examples above, a material forming the first seal, the second seal and the third seal (a) extends along the first longitudinal recess and links the first seal to the second seal and (b) extends along the second longitudinal recess and links the second seal to the third seal.

In another example, a method of forming a valve stem for a suction valve of an endoscope, the method comprises forming a valve stem body from a first material, the valve stem body comprises: a first circumferential recess, a second circumferential recess, and a third circumferential recess, forming a first seal in the first circumferential recess, a second seal in the second circumferential recess, and a third seal in the third circumferential recess; and wherein the first seal, the second seal, and the third seal are formed from a second material and the second material is a different material than the first material.

Alternatively or additionally to any of the examples above, the valve stem body further comprises: a first opening, a second opening, and a lumen extending between the first opening and the second opening, wherein the first circumferential recess is proximal of the of the first opening, and wherein the second circumferential recess and the third circumferential recess are distal of the first opening.

Alternatively or additionally to any of the examples above, the valve stem body further comprises: a first longitudinal recess between the first circumferential recess and the second circumferential recess, and a second longitudinal recess between the second circumferential recess and the third circumferential recess.

In another example, a suction valve assembly for use in an endoscope having a lumen configured to extend into a patient's body cavity may comprise a valve well defining a port configured to be in fluid communication with the lumen, and a valve stem configured to adjust within the valve well, the valve stem comprising a first circumferential seal, a second circumferential seal, a third circumferential seal, a first connection component extending between the first circumferential seal and the second circumferential seal, and a second connection component extending between the second circumferential seal and the third circumferential seal, wherein the valve stem may be configured to fluidly isolate the port from a negative pressure when in a first configuration within the valve well, and wherein the valve stem may be configured to fluidly couple the port with the negative pressure and fluidly isolate the port from atmosphere when in a second configuration within the valve well.

Alternatively or additionally to any of the examples above, the valve stem may further comprise a first opening, a second opening, and a lumen extending between the first opening and the second opening, wherein the first circumferential seal may be proximal of the of the first opening, and wherein the second circumferential seal and the third circumferential seal may be distal of the first opening.

Alternatively or additionally to any of the examples above, the valve stem may further comprise a first circumferential recess at which the first circumferential seal is located, a second circumferential recess at which the second circumferential seal is located, a third circumferential recess at which the third circumferential seal is located, a first longitudinal recess at which the first connection component is located, a second longitudinal recess at which the second connection component is located, and wherein the first seal, the second seal, the third seal, the first connection component, and the second connection component may be a single piece of material.

In a further example, a medical device comprises: a proximal endoscope handle, a distal tip unit adapted to be inserted into a patient's body cavity, an elongate tube extending between the proximal endoscope handle and the distal tip unit, and a suction valve in communication with a lumen of the elongate tube to adjustably fluidly couple the lumen to a negative pressure, the suction valve comprises: a valve well, and a valve stem configured to adjust within the valve well, the valve stem comprising a first circumferential seal, a second circumferential seal, and a third circumferential seal, and wherein the valve stem is configured to fluidly isolate the lumen from the negative pressure when in a first configuration within the valve well, and wherein the valve stem is configured to fluidly couple the lumen with the negative pressure and fluidly isolate the lumen from atmosphere when in a second configuration within the valve well.

Alternatively or additionally to any of the examples above, the valve stem may comprise: a radial opening, an axial opening, a valve stem lumen fluidly coupling the radial opening and the axial opening, and wherein the first circumferential seal is located proximal of the radial opening and the second circumferential seal and the third circumferential seal are located between the radial opening and the axial opening.

Alternatively or additionally to any of the examples above, the valve stem may comprise: a radial opening, an axial opening, a valve stem lumen fluidly coupling the radial opening and the axial opening, and wherein when the valve stem is in the first configuration, the radial opening is in fluid communication with atmosphere and the negative pressure, and wherein when the valve stem is in the second configuration, the radial opening is in fluid communication with the negative pressure and fluidly isolated from atmosphere.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic perspective view of an illustrative suction valve;

FIG. 4 depicts a schematic cross-section view of an illustrative suction valve, with the suction valve in a first configuration;

FIG. 5 depicts a schematic cross-section view of the illustrative suction valve in FIG. 4, with the suction valve in a second configuration;

FIG. 6 depicts a schematic perspective view of an illustrative valve stem body;

FIG. 7 depicts a schematic perspective view of an illustrative valve stem;

FIG. 8 depicts a schematic view of a portion of a valve stem; and

FIG. 9 depicts schematic flow diagram of an illustrative method of making a suction valve.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

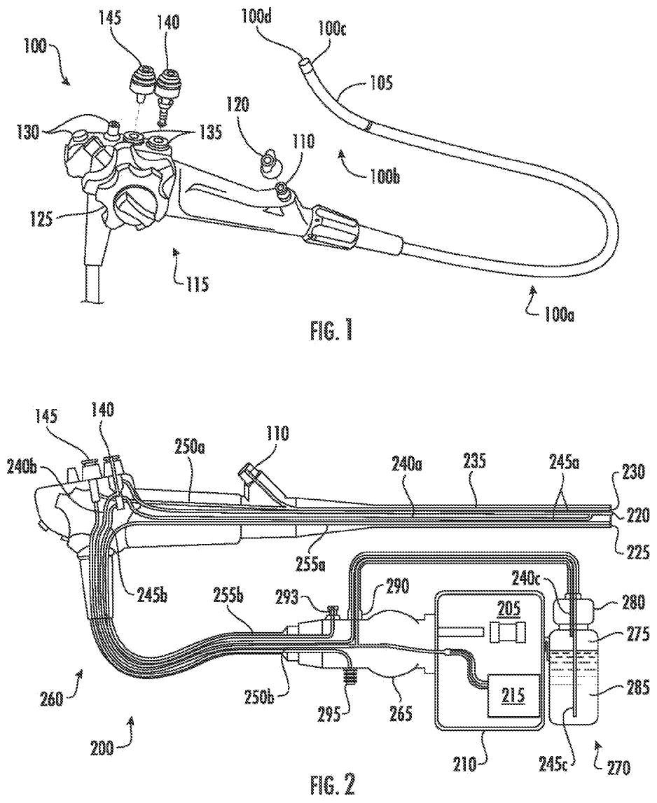

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct suction a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilicus 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position)

In some cases, valve stems of suction valves 145 may be configured to have a close fit with a valve well configured to receive the valve stem in the endoscope 100. In such suction valves 145, when the valve stem is in a first position the close fit blocks a flow path or increases a resistance to flow between the working channel 235 and the suction pump and reduces a resistance to flow between atmosphere and the suction pump. Similarly, when the valve stem is in a second position, the close fit blocks a flow path or increases a resistance to flow between the atmosphere and the suction pump and reduces a resistance to flow between the working channel 235 and the suction pump.

Suction valves 145 configured to block flow using close fits between the valve stem and valve well requires valves stems that are precisely manufactured. The precision required to produce suction valves with close fits requires expensive materials (e.g., metals, etc.), highly precise machinery, and is time consuming to achieve.

Additionally, suction valves 145 with close fit valve stems and valve wells are manufactured to have at least some clearance to allow the valve stem to adjust positions within the valve well. This clearance, may result in leakage during use, which may lead to two issues noticeable by a physician. The first is when the suction valve 145 is in a position intended to block suction from the working channel 235, there is still some suction flow passing through the working channel 235 and the suction valve 145 to the suction pump. The smaller the clearance between the valve stem and the valve well, the less unwanted flow through the working channel 235 that occurs and the larger the clearance, the more unwanted flow through the working channel 235, however, clearance is needed to facilitate movement of the valve stem with in the valve well. When flow is actively moving up the working channel 235 in such configurations of the suction valve 235, users may perceive the suction as “poor insufflation” due to the suction of the suction pump pulling volume from a body lumen in which the user is working, even when the suction valve 145 is in a position intended to block a suction flow from the working channel 235. Second, when a valve stem of the suction valve 145 is in a position within a valve well to facilitate a suction flow between the working channel 235 and the suction pump through the suction valve 145, the flow from atmosphere to the suction pump may not be completely blocked. Any such leaking from atmosphere may reduce a pressure differential between suction valve and the distal end of the working channel 235, which leads to a reduced suction force or negative pressure, reduced flow rates, and aerated flow through the fluid path to the suction pump.

Suction valves 145 configured to operate with close-fit valve stems and valve wells may work well enough when intended for re-use in multiple procedures, as a price point for such suction valves can be high enough to justify manufacturing the suction valves 145 from materials and with the necessary precision that can achieve and maintain desired tolerances over the life of the reusable suction valves 145. However, a price point of a single use suction valve may not allow for use of the necessary materials, tools, and/or precise manufacturing required to achieve and/or maintain tolerances over the life of single-use suction valves.

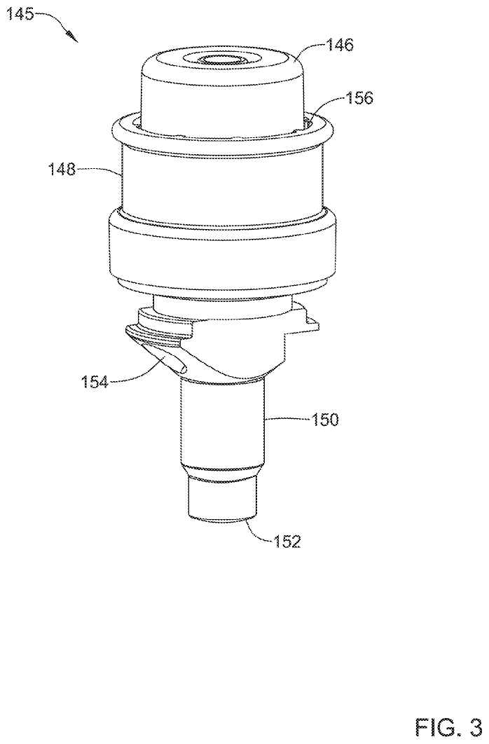

The suction valve configurations for endoscopes 100 and/or other suitable scopes discussed herein address the above-noted concerns with existing suction valves and are configured to mitigate and/or eliminate leakage along an unintended flow path through the suction valve 145. FIG. 3 depicts a schematic perspective view of an illustrative suction valve 145 configured to mitigate and/or eliminate leakage along an unintended flow path through the suction valve 145.

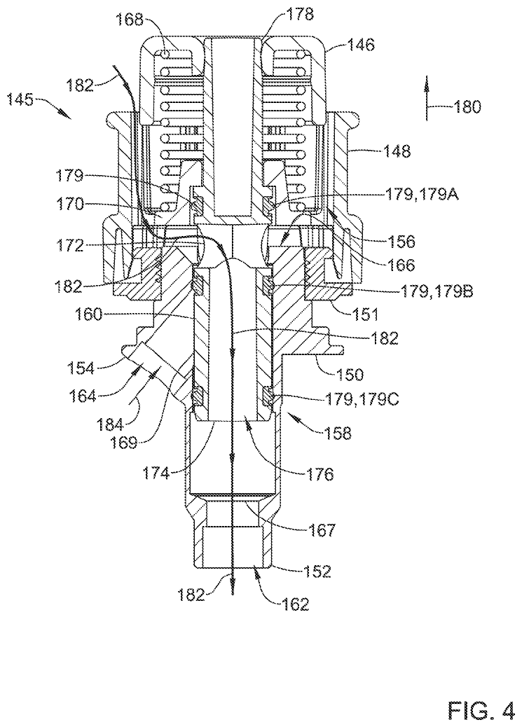

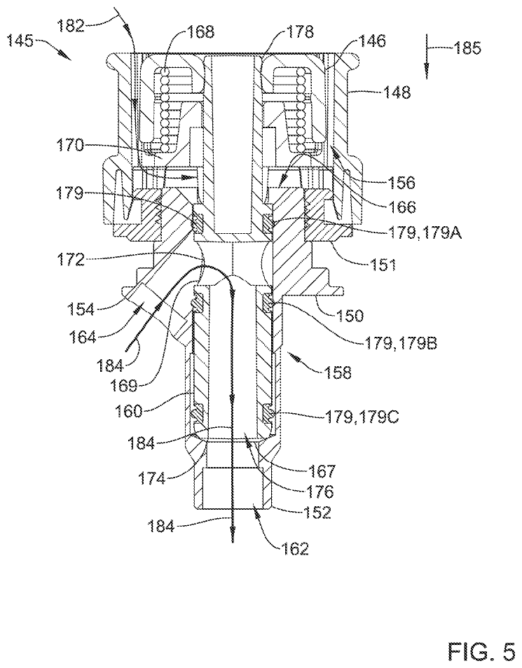

The suction valve 145 depicted in FIG. 3 includes a cap or button 146, a collar 148, and a main body 150, where the main body 150 may define a suction port 152 and a working channel port 154. The collar 148 may define an opening 156 (e.g., a proximal opening of a valve well 158, as depicted in FIGS. 4 and 5, discussed below) for fluid from atmosphere (e.g., air) to flow through the suction valve 145. Although certain components of the suction valve 145 are depicted and described, other suitable components of the suction valve 145 are contemplated and/or certain depicted and described components of the suction valve may be combined to form a single component and/or separated to form multiple components.

In some cases, the collar 148 and the main body 150 may form, be components of, and/or define a valve well 158 (e.g., as depicted in FIGS. 4 and 5, discussed below). Although the collar 148 and the main body 150 are depicted as separate components, the valve well 158 may be formed from a single component, as desired, and incorporate one or more of the features of the collar 148 and the main body 150 discussed herein.

In operation, the suction valve 145 depicted in FIG. 3 may have a first position or configuration (e.g., a relaxed or closed position) and a second position or configuration (e.g., an actuated or opened position). When the suction valve 145 is in the first position, the suction valve is configured to create a fluid path between the opening 156 leading to atmosphere and the suction port 152. When the suction valve 145 is in the second position, the suction valve 145 is configured to create a fluid path between the working channel port 154 and the suction port 152.

FIG. 4 depicts a schematic cross-sectional view of the suction valve 145 depicted in FIG. 3, where the suction valve 145 is in a first position (e.g., a relaxed or closed position). As depicted in FIG. 4, the collar 148 may be coupled to the main body 150. The collar 148 may be directly or indirectly coupled to the main body 150 in any suitable manner including, but not limited to, via adhesives, a threaded connection, a luer lock connection, a snap connection, a ball-detent connector, a friction fit, and/or additional or alternative coupling techniques. In some cases, the collar 148 may be coupled to the main body 150 via an intermediate component 151 threadedly coupled to the main body 150 and coupled to the collar 148 via a snap connection, as depicted in FIGS. 4 and 5, but this is not required.

The main body 150 of the valve well 158 depicted in FIG. 4 may be configured to receive a valve stem 160 in communication with the cap 146. For example, the main body 150 may define a first lumen 162 (e.g., a suction lumen) to or extending from the suction port 152, define a second lumen 164 (e.g., a working channel lumen) to or extending from the working channel port 154, and a main body opening 166 (e.g., a proximal opening of the main body 150) configured to receive the valve stem 160. In some cases, the main body opening 166 may be configured to receive fluid from atmosphere that travels through the opening 156 to atmosphere and into the valve stem 160. In addition to main body opening 166, the valve well 158 may include a distal opening 167 leading to the suction port 152 through the first lumen 162, along with an intermediate opening 169 between the main body opening 166 and the distal opening 167 leading to the working channel port 154 through the second lumen 164. Additionally or alternatively, the valve well 158 may include one or more other suitable openings and/or configurations of openings.

In some cases, the valve well 158 may be configured to facilitate translation (e.g., longitudinal translation and/or rotational translation) of the valve stem 160 in the main body 150 and/or the collar 148 in response to forces acting on the valve stem 160. Further, the valve well 158 may have openings to the first lumen 162 and the second lumen 164, as discussed. In some cases, the valve well 158 may be in fluid communication with and/or define the suction port 152, the first lumen 162, working channel port 154, the second lumen 164, and the main body opening 166, where the translation of the valve stem 160 within the valve well 158 may adjust flow paths through the suction valve 145.

The suction valve 145 may further include one or more biasing mechanisms 168. The biasing mechanisms 168 may be any suitable type of biasing mechanism configured to bias the cap 146 to a first position, allow the cap 146 to adjust to a second position in response to a force applied to the cap 146, and return the cap 146 to the first position when the force applied to the cap 146 is entirely or at least partially removed from the cap 146. In one example, the biasing mechanism 168 may be a spring, as depicted in FIG. 4, but this is not required and other suitable biasing mechanisms are contemplated.

When the suction valve 145 includes the cap 146 and the collar 148, the biasing mechanism 168 may extend between the cap 146 and the valve well 158 (e.g., between the cap 146 and the collar 148, when included) to facilitate the cap 146 adjusting relative to the main body 150 and/or the collar 148. In some cases, the biasing mechanism 168 may extend between the cap 146 and a shelf or shoulder 170 of the collar 148, where the collar 148 may at least partially define the opening 156 to atmosphere. Alternatively or additionally, the biasing mechanism 168 may extend between the cap 146 and/or one or more other suitable components of the suction valve 145 to facilitate the cap 146 adjusting relative to the main body 150.

The valve stem 160 may couple to the cap 146 in any suitable manner. In some cases, a portion 178 (e.g., a proximal portion) of the valve stem 160 extending proximally of the first opening 172 may be coupled to the cap 146 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms.

The valve stem 160 may have any suitable configuration configured to adjust positions within the valve well 158, adjust flow paths to the suction port 152, and couple to the cap 146. In one example, the valve stem 160 may be elongated and define a first opening 172, a second opening 174, and a lumen 176 extending between the first opening 172 and the second opening 174. Further, the valve stem 160 may include the portion 178 extending proximal of the first opening 172 that may be configured to couple to the cap 146.

The first opening 172, the second opening 174, and the lumen 176 may have any suitable configuration configured or otherwise designed to facilitate adjusting flow paths within the suction valve 145 based on a position of the valve stem 160 relative to the valve well 158. In one illustrative configuration, the first opening 172 of the valve stem 160 may be a proximal opening of the valve stem 160. In some cases, the first opening 172 may be a side or radial opening that is in fluid communication with the lumen 176, but this is not required and the first opening 172 may be an axial or end opening in the valve stem 160, as desired. Although not required, the second opening 174 of the valve stem 160 may be a distal opening of the valve stem 160. The second opening 174 may be located at a distal end of the valve stem or at least distally of the first opening 172. In some cases, the second opening 174 may be an axial or end opening in the valve stem 160 that is in fluid communication with the lumen 176, as depicted in FIG. 4, but this is not required and the second opening 174 may be a side opening, as desired.

The valve stem 160 may include one or more seals 179 that are configured to translate with the valve stem 160. In one example, as depicted in FIG. 4, the valve stem 160 may include a first seal 179a, a second seal 179b, and a third seal 179c. The seals 179 may be configured about the valve stem 160 in any suitable manner for interacting with (e.g., contacting) an inner wall or walls of the valve well 158 to create a barrier to fluid flow. Illustratively, the first seal 179a may extend entirely or at least partially circumferentially around the valve stem 160 at a location proximal of the first opening 172 of the valve stem 160, the second seal 179b may extend entirely or at least partially circumferentially around the valve stem 160 at a location distal of the first opening 172, and the third seal 179c may extend entirely or at least partially circumferentially around the valve stem 160 at a location distal of the second seal 179b, but other suitable configurations are contemplated.

The valve stem 160 may be configured to have increased clearance within the valve well 158 when compared to traditional valve stems, and particularly when compared to reusable valve stems. For example, traditional valve stems may be configured to have clearance within a valve well of less than about 0.002 inches, such as a clearance of about 0.002 to about 0.0005 inches, where a body of the valve stem 160 of the present disclosure may have a clearance of at least about 0.005 inches circumferentially from an inner wall of the valve well 158. In one example, the body of the valve stem 160 may be sized to (e.g., configured to) have a clearance of about 0.005 inches to about 0.010 inches from the inner wall of the valve well 158. The ability to form valve stems 160 to have larger clearances within valve wells 158 relative to traditional valve configurations facilitates using less precise manufacturing techniques and/or materials with more variable tolerances to create the suction valve 145 than has traditionally been used for suction valves of endoscopes. Other suitable clearances and/or dimensions are contemplated.

The cross-sectional views of FIG. 4 and FIG. 5 depict an operation of the suction valve 145. As shown in FIG. 4, the valve stem 160 and the suction valve 145 are in an illustrative first configuration (e.g., relaxed or closed position). As shown in FIG. 5, the valve stem 160 and the suction valve 145 are in an illustrative second configuration (e.g., actuated or open position).

When the valve stem 160 and the suction valve 145 are in the illustrative first configuration, a biasing force (e.g., a force acting in a direction of arrow 180) of the biasing mechanism 168 may bias the valve stem 160 in a proximal direction at least partially within the valve well 158 such that the opening 156 to atmosphere, the main body opening 166, and the distal opening 167 are in fluid communication. For example, as shown in FIG. 4, the valve stem 160 may be biased proximally such that a flow path along arrows 182 connects atmosphere with a suction pressure through the opening 156 to atmosphere, the main body opening 166, first opening 172 of the valve stem 160, second opening 174 of the valve stem 160, and the lumen 176 between the first opening 172 and the second opening 174. When the valve stem 160 is in the first configuration, the first seal 179a may engage the collar 148 (when included) and may be proximal of the intermediate opening 169 and the main body opening 166, the second seal 179b may be distal of the main body opening 166 and proximal of the intermediate opening 169 and may contact the inner wall of the valve well 158, the second seal 179b may be a barrier between the main body opening 166 and the intermediate opening 169, the third seal 179c may be distal of the intermediate opening 169 and may contact with the inner wall of the valve well 158, the third seal 179c may be a barrier between the intermediate opening 169 and the distal opening 167, and the intermediate opening 169 and/or fluid therein (represented by arrow 184) may be fluidly isolated from the lumen 176 of the valve stem 160, the distal opening 167, and the suction pressure due to a positioning the second seal 179b, the third seal 179c, and the wall of the valve stem 160.

When the valve stem 160 and the suction valve 145 are in the illustrative second configuration, a force in the direction of arrow 185 (e.g., a direction opposite or substantially opposite the arrow 180) acting on the cap or button 146 works against (e.g., opposes) the biasing force of the biasing mechanism 168 to adjust the valve stem 160 in a distal direction at least partially within the valve well 158 to adjust the valve stem 160 from the first configuration to the second configuration, such that the intermediate opening 159 and the distal opening 167 are in fluid communication. For example, as shown in FIG. 5, the valve stem 160 may be adjusted distally such that a flow path along arrows 184 connects fluid from the working channel of the endoscope with a suction pressure through the working channel port 154, the second lumen 164, the intermediate opening 169, the first opening 172 of the valve stem 160, the second opening 174 of valve stem 160 and the lumen 176 between the first opening 172 and the second opening 174. When the valve stem 160 is in the second configuration, the first seal 179a may be located proximal of the intermediate opening 169 and distal to the main body opening 166, the first seal 179a may be a barrier between the proximal opening or opening 156 to atmosphere and the intermediate opening 169, the third seal 179c may be located distal of the intermediate opening 169 and may be a barrier between the intermediate opening 169 and the distal opening 167, and the proximal opening 156 and/or fluid therethrough from atmosphere (represented by arrow 182) may be fluidly isolated from the lumen 176 of the valve stem 160 and the suction pressure due to a positioning the first seal 179a and a wall of the valve stem 160. In some cases, the valve stem 160 may be configured such that when the valve stem 160 is in the second configuration, the second seal 179b may be located distal of the intermediate opening 169 and one or both of the second seal 179b and the third seal 179c may be a barrier between the intermediate opening 169 and the distal opening 167, but this is not required.

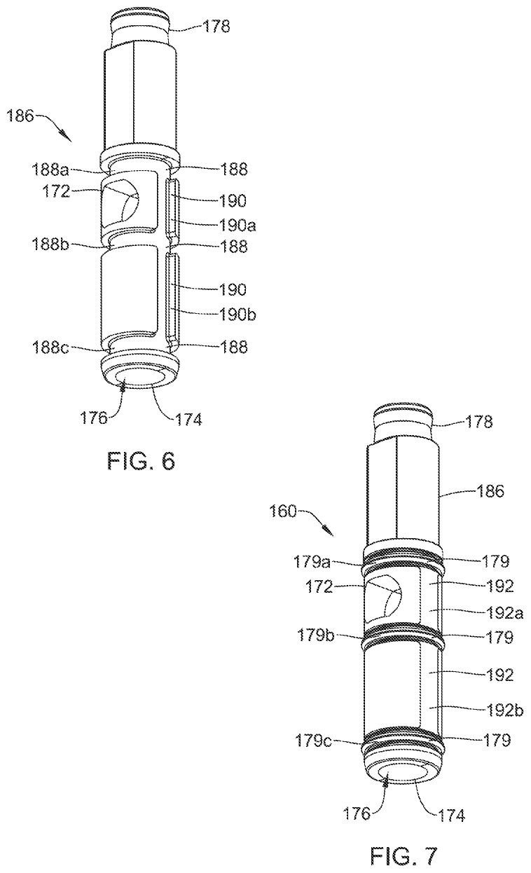

FIG. 6 depicts a perspective view of an illustrative valve stem body 186 (e.g., a body of the valve stem 160 without seals 179 thereon).

The valve stem body 186 may define the first opening 172 of the valve stem 160, the second opening 174 of the valve stem 160, and the lumen 176 extending between the first opening 172 and the second opening 174. Additionally or alternatively, the valve stem body 186 may define one or more recessed portions 188. As depicted in FIG. 6, the valve stem body 186 may define a first recessed portion 188a, a second recessed portion 188b, and a third recessed portion 188c. Although not required, the first recessed portion 188a may be associated with and/or configured to receive at least a portion of the first seal 179a and may be located proximal of the first opening 172, the second recessed portion 188b may be associated with and/or configured to receive at least a portion of the second seal 179b and may be located distal of the first opening 172, and the third recessed portion 188c may be associated with and/or configured to receive at least a portion of the third seal 179c and may be located distal of the second recessed portion 188b.

In some cases, the valve stem body 186 may include one or more recessed portions 190 (e.g., channels or runners) extending between the recessed portions 188 configured to receive the seals 179, as discussed in greater detail below. In one example, a first elongate or longitudinally extending recessed portion 190a may extend between the first recessed portion 188a and the second recessed portion 188b and a second elongate or longitudinally extending recessed portion 190b may extend between the second recessed portion 188b and the third recessed portion 188c. Although the first elongated recessed portion 190a and the second recessed portion 190b are shown as being aligned in FIG. 6, this is not required and the elongate recessed portions 190a, 190b may be offset from one another. Further, although two elongate recessed portions 190a, 190b are depicted in FIG. 6, other suitable numbers of recessed portions 190 connecting recessed portions 188 configured to receive seals 179 are contemplated.

The proximal portion 178 of the valve stem 160 and the valve stem body 186 may take on any suitable configuration. In one example, the proximal portion 178 may be elongated and may have a consistent cross-sectional diameter along its length, as shown in FIGS. 4 and 5. In another example, the proximal portion 178 may have varying diameters along its length to facilitate receiving and/or connecting to the cap or button 146 and/or for one or more other suitable purposes.

The valve stem body 186 may be formed in any suitable manner. In some cases, though not required, the valve stem 160 may be formed using a molding process, an injection molding process, a casting process, a finishing process, sanding, and/or by or with one or more additional or alternative manufacturing techniques. In one illustrative example, the valve stem body 186 may be formed using an injection molding process.

FIG. 7 depicts a perspective of the valve stem 160, where the seals 179 have been formed in and/or over the recess portions 188 depicted in FIG. 6 and configured to span a clearance between the valve stem body 186 and the inner wall of the valve well 158 so as to form a barrier between the valve stem body 186 and the valve well 158 that prevents fluid from crossing the seals 179. The seals 179 may be applied to the valve stem body 186 in any suitable manner. In one example, the seals 179 may be over-molded onto the valve stem body 186 (e.g., in the recessed portions 188) by a suitable process such as an injection molding process. Alternatively or additionally, the seals 179 may be rolled onto the valve stem body 186 and into the recessed portions 188. Other suitable techniques including, but not limited to, co-extrusion are contemplated for forming the valve stem body 186 and the seals 179 of the valve stem 160.

The recessed portions 190 may facilitate an over-molding of the seals 179 in the recessed portions 188. For example, in an over-molding process, the material used for the seals 179 may be applied to the circumferentially extending recess portions 188 via one or more of the elongated recessed portions 190 coupling the circumferentially extending recess portions 188, thereby forming one or more lengths 192 of material (e.g., a first length 192a of material and a second length 192b of material, as depicted in FIG. 7) connecting each of the seals 179 to an adjacent seal 179. The illustrative recessed portions 188, 190 may facilitate material forming the seals 179 to be applied to the valve stem body 186 at a single gate or injection point that is not located at a seal 179. Utilizing a gate or injection point for applying material of the seals 179 to the valve stem body 186 at a location spaced from the seal 179 (e.g., along the recessed portions 190) may facilitate post-processing of the material to smooth out the gate by isolating the post-processing of the gate, if any, to a location spaced from the seals 179. For example, there may be a slight dimple or sight nipple of material at the gate subsequent to injection, which would undesirable be if positioned at the seals.

The valve stem body 186 and the seals 179 may be formed from any suitable materials. In some cases, the valve stem body 186 may be formed from a first material and the seals 179 may be formed from a second material, where the second material may be the same as or different than the first material. The valve stem body 186 may be formed from a hard or rigid polymer and the seal 179 may be formed from a flexible polymer, but this is not required. In one example, the valve stem body 186 may be formed from one or more of metals, polymer, acrylonitrile butadiene styrene (ABS), polycarbonate, and/or other suitable materials. In another example, the seals 179 may be formed from one or more of a polymer, thermoplastic elastomers (TPE), thermoplastic polyurethane (TPU), liquid silicone rubber (LSR), and/or other suitable materials.

The material of the seals 179 may have any suitable durometer. In one example, the material of the seals 179 when formed on the valve stem body 186 may have a durometer in a range of about 20-80 shore A, about 30-60 shore A, and/or other suitable values within one or more other suitable ranges of durometer, but could be softer or firmer depending on the geometry used for the seals and the amount of interference desired with the valve well 158. In one example, the seals 179 may be formed from silicone with a durometer in a range of 40-50 shore A, but this is not required.

The seals 179 may have any suitable shape configured to form a barrier to fluid along a space between the valve stem body 186 and the inner wall of the valve well 158. For example, suitable shapes and/or configurations of the seals 179 may include, but are not limited to, o-rings (e.g., circular cross-sections), X-shaped cross-sectional o-rings, wiper seal rings, disc shaped rings, pre-configured seals applied to the circumferentially extended recesses 188, and/or other suitable shapes and/or configurations of the seals 179.

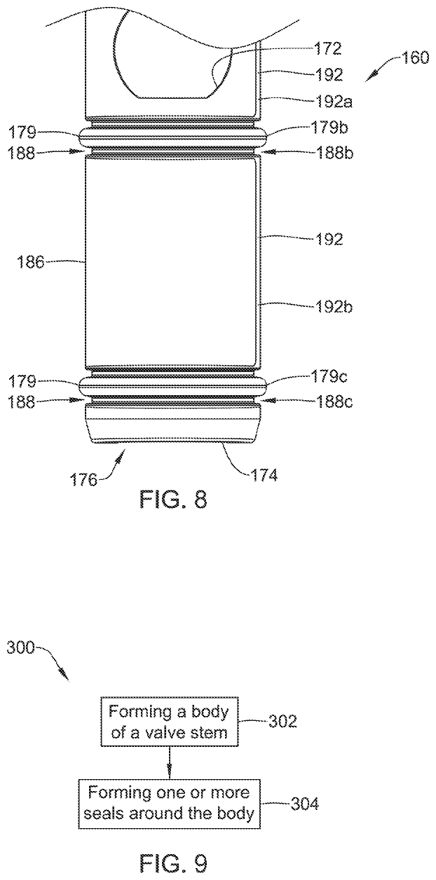

As depicted in FIG. 8, the distal end of the valve stem 160 includes seals 179 that are protruding discs configured to wipe along an inner surface of the valve well 158 as the valve stem 160 translates within the valve well 158. Even so, geometry of the seals could be wider or narrower than the seals 179 depicted in FIG. 8 relative to a width or diameter of the valve stem body 186 and/or the recesses 188 depending on a material selected to form the seals 179 in order to achieve a desired frictional force at an interference with the valve well 158. In some cases, a geometry and/or material of the seals 179 may be configured to minimize a force normal to the wall of the valve well 158, while allowing the seals 179 to maintain a fluid barrier between the valve stem body 186 and the inner wall of the valve well 158, but this is not required.

FIG. 9 depicts a method 300 of forming a valve stem (e.g., the valve stem 160 and/or other suitable valve stem) for an endoscope (e.g., the endoscope and/or other suitable endoscope). The method may include forming 302 a body of a valve stem (e.g., the valve stem body 188 and/or other suitable body of a valve stem) and forming 304 one or more seals (e.g., the seals 179 and/or other suitable seals) around the body. Once the valve stem is formed, the valve stem may be inserted into a valve well and coupled to a cap or button to form a suction valve (e.g., the suction valve 145 and/or other suitable suction valves).

In some cases, forming 302 the body of the valve stem may include forming the body from a first material. Further, the body may include one or more circumferential recesses (e.g., recesses 188 and/or other suitable recesses) configured to receive a seal. In one example, forming 302 the body of the valve stem may include forming a first circumferential recess, forming a second circumferential recess, and forming a third circumferential recess in the body of the valve stem. The recesses may be formed in a single forming step (e.g., using injection molding and/or other suitable manufacturing technique) and/or formed individually, as desired.

In some cases, the recesses may be connected by one or more longitudinally extending recesses (e.g., the elongated recessed portions 190 and/or other suitable recesses) configured to couple circumferential recessed portions to one another. In one example, a first longitudinal recess may be formed between the first circumferential recess and the second circumferential recess and a second longitudinal recess may be formed between the second circumferential recess and the third circumferential recess.

Further, the forming 302 the body of the valve stem may include forming a first opening (e.g., a side or radial opening and/or other suitable opening), a second opening (e.g., an end or terminal opening and/or other suitable opening), and a lumen extending between the first opening and the second opening. When the first and second openings are included in the valve stem body, the first circumferential recess may be formed proximal of the first opening and the second and third circumferential recesses may be formed distal of the first opening.

Forming 304 the seals around the body may include forming a seal in each circumferential recesses of the body. In one example, a first seal may be formed in the first circumferential recess, a second seal may be formed in the second circumferential recess, and a third seal may be formed in the third circumferential recess. In some cases, the seals may be formed around the body by forming a first seal in the first circumferential recess, a second seal in the second circumferential recess, a third seal in the third circumferential recess, a first length of material in the first longitudinal recess that links the first seal to the second seal, and a second length of material in the second longitudinal recess that links the second seal to the third seal. Further, forming 304 the seals may include forming seals from a second material that is different than the first material, but this is not required. In some cases, the second material may be over-molded onto the first material, but this is not required. Alternatively or additionally, the seals may be pre-formed (e.g., as O-rings and/or other suitable pre-formed configurations) and formed around the body by applying the preformed seals to a desired location along the body.

In some cases, the forming of the seals around the body may include forming the seals while forming the body. In one example, the body and the seals of the valve stem may be co-extruded and molded with one or more materials. In another example, the valve stem may be monolithic such that the body and seals are formed from a single material using molding and/or machining techniques.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A suction valve assembly for a medical device, comprising:
a valve well having a proximal opening, a distal opening, and an intermediate opening positioned between the proximal opening and the distal opening;
a valve stem configured to translate within the valve well, the valve stem comprising:
a first opening;
a second opening; and
a lumen extending from the first opening to the second opening;
a first seal extending circumferentially around the valve stem at a location proximal of the first opening;
a second seal extending circumferentially around the valve stem at a location distal of the first opening; and
a third seal extending circumferentially around the valve stem at a location distal of the second seal; and
wherein the valve stem has a first configuration in the valve well in which the second seal is a barrier between the proximal opening and the intermediate opening, the third seal is a barrier between the intermediate opening and the distal opening, and the lumen is fluidly isolated from the intermediate opening;
wherein the valve stem has a second configuration in the valve well in which the first seal is proximal the intermediate opening and the lumen is fluidly isolated from the proximal opening and the first seal is a barrier between the proximal opening and the intermediate opening.

2. The suction valve assembly of claim 1, wherein when the valve stem is in the first configuration, the proximal opening and the distal opening are in fluid communication through the lumen of the valve stem.

3. The suction valve assembly of claim 1, wherein when the valve stem is in the second configuration, the intermediate opening and the distal opening are in fluid communication through the lumen of the valve stem.

4. The suction valve assembly of claim 1, wherein when the valve stem is in the first configuration, the third seal is distal of the intermediate opening and the distal opening is fluidly isolated from the intermediate opening.

5. The suction valve assembly of claim 1, wherein the second seal and the third seal are configured to contact an inner wall of the valve well when the valve stem is in the first configuration.

6. The suction valve assembly of claim 1, wherein the first opening is a radial opening in the valve stem and the second opening is an axial opening in the valve stem.

7. The suction valve assembly of claim 1, wherein the valve well further comprises:
a main body defining the intermediate opening and the distal opening; and
a collar couplable to the main body, the collar defining the proximal opening.

8. The suction valve assembly of claim 7, wherein the first seal is configured to engage the collar when the valve stem is in the first configuration.

9. The suction valve assembly of claim 1, further comprising:
a cap coupled to the valve stem;
a bias mechanism extending between the cap and the valve well; and
wherein the bias mechanism is configured to bias the valve stem to the first configuration.

10. The suction valve assembly of claim 9, wherein the cap, the bias mechanism, the valve stem, and the valve well are configured such that actuation of the cap in a direction opposite a direction of a bias force of the bias mechanism is configured to adjust the valve stem from the first configuration to the second configuration.

11. The suction valve assembly of claim 1, wherein the valve stem comprises:
a first circumferentially recessed portion at which the first seal is located;
a second circumferentially recessed portion at which the second seal is located; and
a longitudinal recessed portion extending between the first circumferentially recessed portion and the second circumferentially recessed portion.

12. The suction valve assembly of claim 1, wherein the valve stem comprises:
a first circumferentially recessed portion at which the first seal is located;
a second circumferentially recessed portion at which the second seal is located;
a third circumferentially recessed portion at which the second seal is located;
a first longitudinal recess extending between the first circumferentially recessed portion and the second circumferentially recessed portion; and
a second longitudinal recess extending between the second circumferentially recessed portion and the third circumferentially recessed portion.

13. The suction valve assembly of claim 12, wherein a material forming one or more of the first seal, the second, seal, and the third seal extends within one or both of the first longitudinal recess and the second longitudinal recess.

14. The suction valve assembly of claim 12, wherein a material forming the first seal, the second seal and the third seal (a) extends along the first longitudinal recess and links the first seal to the second seal and (b) extends along the second longitudinal recess and links the second seal to the third seal.

15. A suction valve assembly for use in an endoscope having a lumen configured to extend into a patient's body cavity, the suction valve assembly comprising:
a valve well defining a port configured to be in fluid communication with the lumen; and
a valve stem configured to adjust within the valve well, the valve stem comprising a first circumferential seal, a second circumferential seal, a third circumferential seal, a first connection component extending between the first circumferential seal and the second circumferential seal, and a second connection component extending between the second circumferential seal and the third circumferential seal; and
wherein the valve stem is configured to fluidly isolate the port from a negative pressure when in a first configuration within the valve well; and
wherein the valve stem is configured to fluidly couple the port with the negative pressure and fluidly isolate the port from atmosphere when in a second configuration within the valve well.

16. The suction valve assembly of claim 15, wherein the valve stem further comprises:
a first opening, a second opening, and a lumen extending between the first opening and the second opening;
wherein the first circumferential seal is proximal of the of the first opening; and
wherein the second circumferential seal and the third circumferential seal are distal of the first opening.

17. The suction valve assembly of claim 15, wherein the valve stem further comprises:
a first circumferential recess at which the first circumferential seal is located;
a second circumferential recess at which the second circumferential seal is located;
a third circumferential recess at which the third circumferential seal is located;
a first longitudinal recess at which the first connection component is located;
a second longitudinal recess at which the second connection component is located; and
wherein the first circumferential seal, the second circumferential seal, the third circumferential seal, the first connection component, and the second connection component are a single piece of material.

18. A medical device comprising:
a proximal endoscope handle;
a distal tip unit adapted to be inserted into a patient's body cavity;
an elongate tube extending between the proximal endoscope handle and the distal tip unit; and
a suction valve in communication with a lumen of the elongate tube to adjustably fluidly couple the lumen to a negative pressure, the suction valve comprises:
a valve well; and
a valve stem configured to adjust within the valve well, the valve stem comprising a first circumferential seal, a second circumferential seal, and a third circumferential seal; and
wherein the valve stem is configured to fluidly isolate the lumen from the negative pressure when in a first configuration within the valve well; and
wherein the valve stem is configured to fluidly couple the lumen with the negative pressure and fluidly isolate the lumen from atmosphere when in a second configuration within the valve well.

19. The medical device of claim 18, wherein the valve stem comprises:
a radial opening;
an axial opening;
a valve stem lumen fluidly coupling the radial opening and the axial opening; and
wherein the first circumferential seal is located proximal of the radial opening and the second circumferential seal and the third circumferential seal are located between the radial opening and the axial opening.

20. The medical device of claim 18, wherein the valve stem comprises:
a radial opening;
an axial opening;
a valve stem lumen fluidly coupling the radial opening and the axial opening; and
wherein when the valve stem is in the first configuration, the radial opening is in fluid communication with atmosphere and the negative pressure; and
wherein when the valve stem is in the second configuration, the radial opening is in fluid communication with the lumen and fluidly isolated from atmosphere.
